(19) United States
(12) Patent Application Publication
(10) Pub. No.: US 2019/0256278 A1
(43) Pub. Date: Aug. 22, 2019

(54) COMPRESSED HAIR SPRAY

(71) Applicant: Conopco, Inc., d/b/a UNILEVER, Englewood Cliffs, NJ (US)

(72) Inventors: Gillian BROWN, Heswall, Wirral (GB); Laura FRANKLIN, Liverpool (GB); Llyr Glyndwr GRIFFITHS, Rhos-on-Sea (GB); Leslie Joseph Luke JOINSON, Preton, Wirral (GB); Jonathan Douglas William SMITH, Northwich (GB); Paul James SMITH, Hoylake, Wirral (GB)

(73) Assignee: Conopco, Inc., d/b/a UNILEVER, Englewood Cliffs, NJ (US)

(21) Appl. No.: 16/342,105

(22) PCT Filed: Oct. 11, 2017
(86) PCT No.: PCT/EP2017/075918

§ 371 Date: Apr. 15, 2019

(30) Foreign Application Priority Data
Oct. 19, 2016 (EP) 16194550.6

(51) Int. Cl. B65D 83/14; B65D 83/20; B05B 1/34; A61K 8/04; A61Q 5/06

(52) U.S. Cl. CPC B65D 83/752; B65D 83/205; A61Q 5/06; A61K 8/046; B05B 1/3436

(57) ABSTRACT

An aerosol hairspray product includes a pressurisable container having a propellant and a container wall which encloses a reservoir, a hairstyling formulation having from 20 wt % to 75 wt % water, by total weight of the hairstyling formulation and propellant and from 1.0 wt % to 15 wt % hairstyling polymer, by total weight of the hairstyling formulation and propellant, and a spraying device attached to the pressurisable container and having a valve and an actuator. The reservoir is configured to store the hairstyling formulation. The valve has a spring means, a valve body having a stem, a restricted tail piece (RTP) having a first diameter of from 0.1 mm to 2.5 mm, and a vapour phase tap (VPT) having a second diameter of from 0.1 mm to 1.5 mm. The actuator has a main spray channel and communicates with an insert.

### FIELD OF INVENTION

The present invention relates to a “compressed” aerosol hairspray product provided in a can having reduced amount of hair styling composition yet providing the same number of uses as a non-compressed product.

### BACKGROUND AND PRIOR ART

Hairstyling products such as hairsprays are used for achieving different hairstyles and for holding hair strands in place for a period of time. Typically, hairsprays comprise film-forming polymers, which when applied to keratin-containing fibres, such as human hair, form fibre-fibre welds. These welds ‘glue’ the fibres together and hence impart hold to the hairstyle.

Aerosol hairspray products usually comprise a pressure- resistant container, a nozzle, a propellant, and a hairstyling formulation. A hairspray composition is normally ejected from such products via aerosol-forming nozzle. See, for example, US2009/0104138A1.

Environmental sustainability has become a matter of growing interest in aerosol applications, and concentrates have the potential to benefit both manufacturers and the environment. Aerosol concentrates offer manufacturers cost advantages in terms of reduced packaging (both primary and secondary), reduced propellant use, reduced shipping costs, and the like. The environment is benefited by a product that, compared to conventional aerosols, emits less propellant (e.g., volatile organic compound (VOC)), generates less packaging waste and, from manufacture through disposal, has a reduced carbon footprint.

It is, therefore, desirable to reduce the size and volume of pressurised products, such as hairsprays. Such products reduce the amount of hair spray composition per consumer use without impacting performance, thus maintaining the same number of consumer uses per can.

GB 2,299,507 A (Unilever) discloses low flow rate propellant driven antiperspirant compositions comprising less than 60% by weight of a propellant and an initial spray rate of no more than 0.5 g/s.

EP 674,899 B1 (Unilever) discloses concentrated deodorant compositions comprising propellant, most preferably at 30-60% by weight of the composition, and having a discharge valve adapted to allow the composition to be sprayed at an initial spray rate of less than 0.3 g/s.

US2015/0004200 (The Procter & Gamble Company) discloses an aerosol hairspray product with a pressurisable container containing an aqueous, substantially alcohol free hairstyling formulation. A spraying device is attached to the container for dispensing the hairstyling formulation and comprises a valve and a nozzle, wherein the valve comprises a valve body, a stem and a spring means. The valve body houses an insert having an insert orifice and at least two channels, which are tangentially disposed about the insert orifice. The valve body also comprises at least two vapour taps, and the insert orifice is capable of being in liquid communication with the hairstyling formulation in the reservoir.

Consumer habits and perceptions can be exceedingly difficult to change. If a product does not fit conventional spray habits, consumer acceptance of that product may be difficult, no matter how good the product. Conversely, a product that matches consumer behaviour but does not deliver on efficacy and sensory requirements is unlikely to be commercially accepted. When providing aerosol products in concentrated form, reference need be made to the manner in which the aerosol is conventionally used. A typical spray period for a hair spray product, by which is meant the time during which an actuator is actively engaged to dispense product, is typically on the order of five seconds.

Producing an acceptable aerosol concentrate requires more than simply changing the level of active, since irrespective of active level, consumers are likely to stick to their conventional application habits. Minimizing spray rate is another approach to concentrates. Typical spray rates for hair spray aerosols are of the order of 0.5 g/s to 1.5 g/s, but lower rates may be desirable for concentrated aerosols. Minimizing spray rate theoretically requires little or no disruption to consumer behavior, because the same dose can then be applied in approximately the same time, despite the aerosol being more concentrated. However, reducing spray rate presents numerous issues for product engineers. As a practical matter, reducing spray rate can exacerbate nozzle clogging and increase spluttering. Additionally, reduced spray rates present challenges in terms of providing compositions that provide acceptable sensory properties, given the confines of consumer application habits and typical application spray periods. Reduced spray rate can also result in reduced product efficacy if insufficient active is dispensed. Therefore, compression not only requires reduced spray rate but also changes to the formulation to ensure that the compressed product is acceptable to the consumer.

One method of achieving compression of spray products is to reduce the spray rate. If the spray rate is reduced, it follows that the concentration of the resin in the formulation should be doubled to compensate. However, doubling the resin in the formulation can lead to significant challenges in terms of stability because there is too much solid content. In addition it can lead to clogging issues in the valve, insert and actuator orifice. These all negatively impact product performance and integrity.

There remains a need for concentrated aerosol hair spray products that provide a combination of desirable sensory properties and dispensing properties, without compromising product efficacy. Such products reduce the amount of hair spray composition per consumer use without impacting performance, thus maintaining the same number of consumer uses per can.

We have now found that a specially developed valve can control the spray rate to achieve compression, in combination with an optimised insert, to achieve the correct spray characteristics of a hair styling formulation. Surprisingly, there is no need to double the resin concentration.

Definition of the Invention

In a first aspect of the invention there is provided an aerosol hairspray product, which comprises:

a) a pressurisable container comprising
i) a container wall which encloses a reservoir for storing a hairstyling formulation, and
ii) a propellant;

b) a hairstyling formulation comprising:
(iii) from 20 wt % to 75 wt % water, by total weight of the hairstyling formulation and propellant; and
(iv) from 1.0 wt % to 15 wt % hairstyling polymer, by total weight of the hairstyling formulation and propellant; and

c) a spraying device attached to the container for dispensing the hairstyling formulation from the reservoir of the container, which comprises a valve and an actuator,

wherein the valve comprises a valve body, said valve body comprising a stem and a spring means; and wherein the valve comprises a restricted tail piece (RTP) of diameter 0.1 mm to 2.5 mm, and a vapour phase tap (VPT) of diameter 0.1 mm to 1.5 mm;

wherein the actuator comprises a main spray channel and communicates with an insert, said insert comprising an exit orifice and from 1 to 8 sub-spray channels; wherein said channels are tangentially disposed about the exit orifice;

wherein the exit orifice is capable of being in liquid communication with the hairstyling formulation in the reservoir;

characterised in that the exit orifice has a diameter of from 0.01 to 2 mm; and an orifice thickness of from 0.1 to 1.5 mm;

and wherein the sub-spray channels have a height of from 0.1 to 0.8 mm and a length of from 0.1 to 0.5 mm.

In a second aspect of the invention there is provided a method of treating hair with a hairspray product of the aspect of the invention, comprising the step of applying to the hair a hair styling formulation as defined in the first aspect of the invention, using a hair spray product of the first aspect of the invention.

### BRIEF DESCRIPTION OF THE FIGURES

Embodiments of the invention will now be described with reference to the following non-limiting drawings in which:

FIG. 1 is a perspective view of a hairspray product in accordance with the invention.

FIG. 2 is a cross section view of a spraying device (4) used in the hairspray product of the invention.

FIG. 3 is a cross section view of a valve (5) used in the hairspray product of the invention, where the valve is installed in the container (seated in a valve cup (16)).

FIG. 4(a) is a cross section view of an insert (6) as used in the hairspray product of the invention.

FIG. 4(b) is a cross sectional view of an insert (6) for use in the hairspray of the invention.

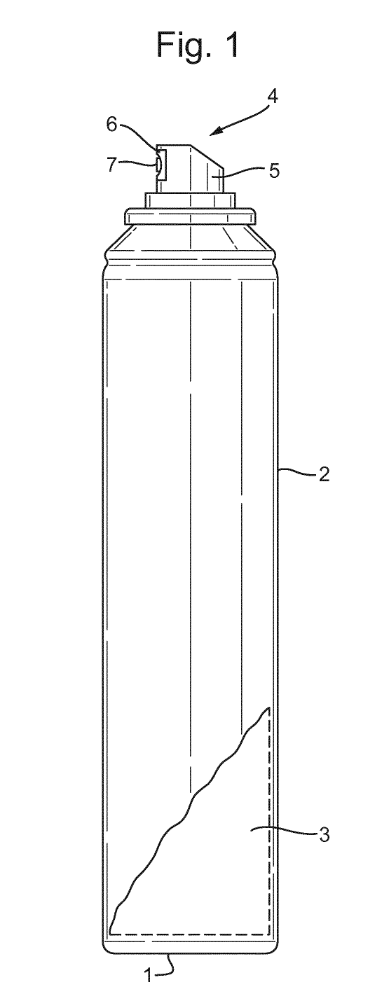

FIG. 1 is a perspective view of a hairspray product comprising a pressurisable container (1) having a container wall (2) and a reservoir (3); with a spraying device (4) comprising an actuator (5) having an insert (6) with an exit orifice (7).

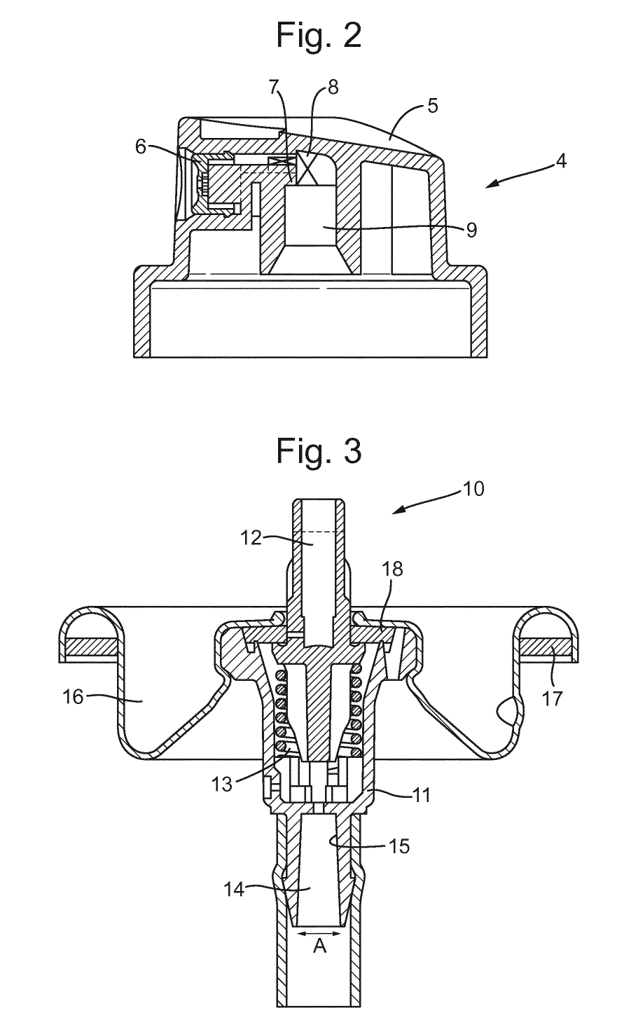

FIG. 2 is a cross section view of a spraying device (4) comprising an actuator (5) having a main spray channel (8) and an insert (6) with an exit orifice (7). A stem socket is also shown.

FIG. 3 is a cross sectional view of a valve (10) used in the hairspray product of the invention, where the valve is installed in the container, seated in a valve cup (16). The valve (10) comprises a valve body (11), said valve body comprising a stem (12) and a spring means (13); and wherein the valve has a restricted tail piece (RTP) (14) with a diameter indicated by a double ended arrow “A”, and a vapour phase tap (VPT) (15). The valve cup (16) comprises an outer gasket (17) and an inner gasket (18).

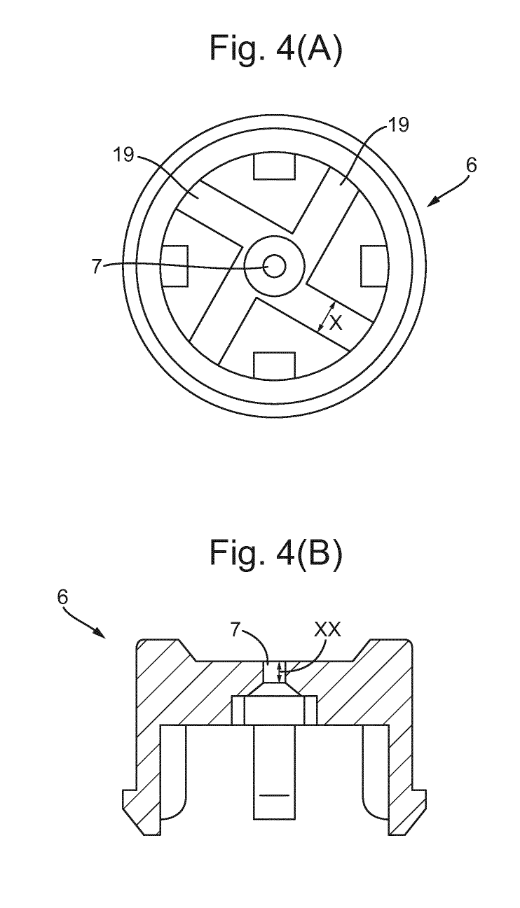

FIG. 4a is a cross section view of an insert (6) comprising an exit orifice (7) and four sub-spray channels (19); wherein said channels are tangentially disposed about the exit orifice (7). Double ended arrow “X” indicates the length of the sub-spray channel as defined in claim 1.

FIG. 4b is a cross section view of an insert (6) showing the exit orifice (7). Double ended arrow “XX” indicates the orifice thickness as defined in claim 1.

General Description of the Invention

The Aerosol Hairspray Product

The product relates to an aerosol hairspray product comprising: a container comprising a reservoir for a hairstyling formulation and a liquefied gas propellant, and a spraying device attached to the container.

In at least one embodiment, the product comprises from 50% to 65% hairstyling formulation, or from 55% to 60% hairstyling formulation, by total weight of the hairstyling formulation and propellant.

In at least one embodiment, the product comprises from 35% to 50% propellant, or from 40% to 45% propellant, by total weight of the hairstyling formulation and propellant.

As used herein, unless otherwise stated, details of the hairstyling formulation refers to the formulation before it is placed into the container whereupon it will mix to an extent with the propellant since the propellant is a liquefied gas propellant. The same is true where details of the propellant are mentioned, unless otherwise stated.

The product is an aerosol hairspray product and thus does not include mousse products or any pump spray products.

Conventional spray rates are typically in the range of from 0.5 to 1.5 g/sec. The product of the invention delivers 80 to 20% of this range, preferably 70 to 30%, most preferably from 60 to 40% of this spray rate. This reduction in spray rate without loss of performance is one of the advantages of this invention. Therefore, the aerosol hairspray product sprays at a delivery rate of from 80 to 20%, preferably from 70 to 30%, most preferably from 60 to 40% of 0.5 to 1.5 g/sec.

The Pressurisable Container

The pressurisable container comprises a container wall which encloses a reservoir for storing a hairstyling formulation, and a propellant.

The container wall may be predominantly straight, curved or tapered.

The container can be any size, but is preferably a “compressed” size or travel size.

The container preferably has a volume (so-called over fill capacity) of from 200 to 300 ml, more preferably from 220 to 280 ml, most preferably from 230 to 260 ml.

The container preferably has a diameter of from 35 to 50 mm, more preferably from 40 to 45 mm.

The container preferably has a height of from 80 to 220 mm, preferably from 90 to 180 mm, most preferably from 110 to 150 mm.

The Propellant

The hairspray composition further comprises a propellant. Preferred propellants are selected from hydrofluorocarbons (for example hydrofluorocarbon 152A), dimethylether, hydrocarbons such as propane, isobutane, butane and mixtures thereof, and compressed gasses such as nitrogen and carbon dioxide and mixtures thereof.

The Hairstyling Formulation

The product comprises a hairstyling formulation comprising a hair styling polymer and a neutraliser in an aqueous composition. It is well known in the art that alcoholic based composition can lead to perceived consumer negative performance such as hair dryness. These negatives are not experienced with aqueous compositions.

The Hair Styling Polymer

The hairstyling polymer is present in an amount of from 1.0 to 10 wt %, preferably from 1.0 to 8.0 wt %, most preferably from 2 to 7 wt % by total weight of the hairstyling formulation and propellant.

The hairstyling polymer may be selected from a VA/Crotonates/Vinyl Neodecanoate Copolymer , Polyurethane-14 (and) AMP-Acrylates Copolymer (for example, DynamX available from AzkoNoble), Butyl Ester of PVM/MA Copolymer (for example, Gantrez available from Ashland), Polyester-5, Octylacrylamide/Acrylates/Butylaminoethyl Methacrylate Copolymer, imidized isobutylene/maleic anhydride copolymer (for example, Aquaflex™ FX-64 available from Ashland), Polyvinylpyrrolidone, Acrylates/Hydroxyesters Acrylates Copolymer (for example, Acudyne 180 available from DOW), Polyurethane-6, Acrylates/Octylacrylamide Copolymer and Acrylates Copolymer (for example, Balance CR available from AkzoNoble and Luvimer 100 P available from BASF), and more preferably from VA/Crotonates/Vinyl Neodecanoate Copolymer (for example, Resyn 28-2930 available from AkzoNoble) and Octylacrylamide/Acrylates/Butylaminoethyl Methacrylate Copolymer (for example, Amphomer available from AkzoNoble).

Preferably the styling resin comprises VA/Crotonates/Vinyl Neodecanoate Copolymer (for example, Resyn 28-2930 available from AkzoNoble) and Octylacrylamide/Acrylates/Butylaminoethyl Methacrylate Copolymer (for example, Amphomer available from AkzoNoble).

The Neutralizer

The neutralizer provides an alkaline base.

The neutralizer is preferably selected from potassium hydroxide, sodium hydroxide, triisopropanolamine, 2-aminobutanol, 2-aminomethyl propanol, aminoethylpropandiol, dimethyl stearamine, sodium silicate, tetrahydroxypropyl ethylenediamine, ammonia, triethanolamine, trimethylamine, aminomerhylpropandiol, and more preferably 2-aminomethyl propanol, dimethyl stearamine and 2-aminobutanol and mixtures thereof.

Aqueous Composition

The water is present in an amount of from 20 wt % to 75 wt % by total weight of the hairstyling formulation and propellant, preferably from 30 wt % to 70 wt %, more preferably from 35 wt % to 60 wt %, most preferably from 35 wt % to 55 wt %, by total weight of the hairstyling formulation and propellant.

The hairstyling formulation preferably contains less than 20 wt % alcohol, more preferably less than 10 wt %, even more preferably 5 wt % and most preferably is substantially free of alcohol.

Additional Ingredients

The formulation may also comprise other adjuncts commonly used in hair sprays. Preferably perfume, silicone (for example phenyl trimethicone, cyclopentylsiloxane, PEG/PPG-20/15 Dimethicone), emotives, corrosion inhibitors, emollients, UV absorbers (for example benzopheonone-4, ethyl hexyl methoxy cinnamate) and mixtures thereof.

The Spraying Device

Attached to the container is a spraying device for spraying the hairstyling formulation onto a substrate.

Valve

The spraying device comprises a valve.

The valve comprises a valve body, said valve body comprising a stem and a spring means.

The valve comprises a restricted tail piece (RTP) of diameter 0.1 mm to 2.5 mm, preferably 0.2 to 1.5 mm, most preferably 0.3 to 1.0 mm.

The valve comprises a vapour phase tap (VPT) of diameter 0.1 mm to 1.5 mm, preferably 0.2 to 1.0 mm, most preferably 0.3 to 0.8 mm.

When installed in the container, the valve is preferably seated in a valve cup (16). The valve cup (16) preferably comprises an outer gasket (17), which enables connection to the can by crimping onto a can bead; and an inner gasket (18). The inner gasket controls the release product when the actuator is pressed.

Actuator

The spraying device comprises an actuator. The actuator typically sits over the stem.

The actuator comprises a main spray channel and communicates with an insert.

Insert

The insert comprises an exit orifice and from 1 to 8 sub-spray channels, preferably 4 to 6 sub-spray channels, most preferably 6 sub-spray channels; wherein said channels are tangentially disposed about the exit orifice.

Suitable inserts are available from, for example, Coster.

The insert controls the cone angle. A wide cone angle is preferred whilst maintaining the desirable particle size distribution.

Channels

The sub-spray channels have a height of from 0.1 to 0.8, preferably from 0.3 to 0.7 mm and a length of from 0.1 to 0.5, preferably from 0.25 to 0.35 mm.

Exit Orifice

The exit orifice is capable of being in liquid communication with the hairstyling formulation in the reservoir.

The exit orifice has a diameter of from 0.01 to 2 mm, most preferably 0.5 mm; and an orifice thickness of from 0.1 to 1.5 mm, preferably from 0.3 to 1.0 mm;

The orifice may be straight or conical in shape.

### EXAMPLES

A hair spray in accordance with the invention and a comparative hair spray are given in Table 1 below.

TABLE 1

| | Comparative Hair | Inventive Hair |
|---|---|---|
| | Spray A | Spray 1 |
| Spraying Device | | |
| Valve RTP | 0.33 mm | 0.8 mm |
| Valve VPT | None | 0.5 mm |
| Valve STEM | 0.33 mm | 0.3 mm |
| Actuator (type & | Aptar | Coster |
| orifice) | 0.64 mm | 0.5 mm |
| Actuator No. of channels | n/a | 6 |
| Spray rate | 100% | 50% |
| Hairstyling Formulation | | |
| Total hairstyling Polymer | 4-5% | 5.5% |
| Water | 53% | 53% |
| Propellant | 40% | 40% |
| Minors (fragrance, pH | 2-3% | 1.5% |
| modifiers) | | |

What is claimed is:

1. An aerosol hairspray product comprising:
a pressurisable container having a propellant and a container wall which encloses a reservoir;
a hairstyling formulation having:
from 20 wt % to 75 wt % water, by total weight of the hairstyling formulation and the propellant; and
from 1.0 wt % to 15 wt % hairstyling polymer, by total weight of the hairstyling formulation and the propellant; and
a spraying device attached to the pressurisable container and having a valve and an actuator,
wherein the reservoir is configured to store the hairstyling formulation;
wherein the valve has a spring means, a valve body having a stem, a restricted tail piece (RTP) having a first diameter of from 0.1 mm to 2.5 mm and a vapour phase tap (VPT) having a second diameter of from 0.1 mm to 1.5 mm;
wherein the actuator has a main spray channel and communicates with an insert, having an exit orifice and from 1 to 8 sub-spray channels;
wherein the sub-spray channels are tangentially disposed about the exit orifice;
wherein the exit orifice is capable of being in liquid communication with the hairstyling formulation in the reservoir;
wherein the exit orifice has a third diameter of from 0.01 mm to 2.0 mm and an orifice thickness of from 0.1 mm to 1.5 mm; and
wherein the sub-spray channels have a height of from 0.1 mm to 0.8 mm and a length of from 0.1 mm to 0.5 mm.

2. The aerosol hairspray product of claim 1, wherein the hairstyling formulation also has less than 20 wt % alcohol, by total weight of the hairstyling formulation and the propellant.

3. The aerosol hairspray product of claim 1, wherein the hairstyling formulation is substantially free from alcohol.

4. The aerosol hairspray product of claim 1, wherein the hairstyling polymer is present in an amount of from 1.0 wt % to 10 wt %, by total weight of the hairstyling formulation and the propellant.

5. The aerosol hairspray product of claim 1, wherein the hairstyling polymer includes at least one of a VA/Crotonates/Vinyl Neodecanoate Copolymer, a Polyurethane-14 AMP-Acrylates Copolymer, a Butyl Ester of PVM/MA Copolymer, a Polyester-5, an Octylacrylamide/Acrylates/Butylaminoethyl Methacrylate Copolymer, an Acrylates/Hydroxyesters Acrylates Copolymer, ora Polyurethane-6, Acrylates/Octylacrylamide Copolymer and Acrylates Copolymer.

6. The aerosol hairspray product of claim 1, wherein the hairstyling formulation also has an alkaline base.

7. The aerosol hairspray product of claim 1, wherein the first diameter is from 0.2 mm to 1.5 mm.

8. The aerosol hairspray product of claim 1, wherein the second diameter is from 0.2 mm to 1.0 mm.

9. The aerosol hairspray product of claim 1, wherein the pressurisable container has a volume of from 200 ml to 300 ml.

10. The aerosol hairspray product of claim 1, wherein the insert has 6 sub-spray channels.

11. The aerosol hairspray product of claim 1, wherein the height is from 0.3 mm to 0.7 mm.

12. The aerosol hairspray product of claim 1, wherein the length is from 0.25 mm to 0.35 mm.

13. The aerosol hairspray product of claim 1, wherein the orifice thickness is from 0.3 mm to 1.0 mm 0.1 to 1.5 mm.

14. (canceled)

15. A method of treating hair with a hairspray product that includes a pressurisable container having a propellant and a container wall which encloses a reservoir, a hairstyling formulation having from 20 wt % to 75 wt % water, by total weight of the hairstyling formulation and the propellant, and from 1.0 wt % to 15 wt % hairstyling polymer, by total weight of the hairstyling formulation and the propellant, and a spraying device attached to the pressurisable container and having a valve and an actuator, the reservoir being configured to store the hairstyling formulation, the valve having a spring means, a valve body having a valve stem, a restricted tail piece having a first diameter of from 0.1 mm to 2.5 mm, and a vapour phase tap having a second diameter of from 0.1 mm to 1.5 mm, the actuator having a main spray channel and communicating with an insert having an exit orifice that is capable of being in liquid communication with the hairstyling formulation in the reservoir and from 1 to 8 sub-spray channels that are tangentially disposed about the exit orifice, the exit orifice having a third diameter of from 0.01 mm to 2.0 mm and an orifice thickness of from 0.1 mm to 1.5 mm, and the sub-spray channels having a height of from 0.1 mm to 0.8 mm and a length of from 0.1 mm to 0.5 mm, the method comprising:
applying the hairstyling formulation to hair using the hairspray product.
